(19) United States
(12) Patent Application Publication
(10) Pub. No.: US 2026/0358310 A1
(43) Pub. Date: Nov. 12, 2026

(54) METHOD OF PRODUCING AN ANALYSIS DEVICE, ANALYSIS DEVICE, ANALYSIS ARRANGEMENT AND ANALYSIS METHOD

(71) Applicant: UNIVERSITY OF TARTU, Tartu (EE)

(72) Inventors: Hanno EVARD, Tartu (EE); Indrek SAAR, Tartu (EE); Airiin LAANEVÄLI, Tartu (EE)

(21) Appl. No.: 19/139,211

(22) PCT Filed: Dec. 14, 2023
(86) PCT No.: PCT/EP2023/085913

§ 371 Date: Jun. 13, 2025

(30) Foreign Application Priority Data
Dec. 16, 2022 (GB) 2219073.0

(51) Int. Cl. G01N 30/91; G01N 30/92; G01N 33/558

(52) U.S. Cl. CPC G01N 30/91; G01N 30/92; G01N 33/558

(57) ABSTRACT

An analysis device for analyzing a liquid sample for one or more analytes includes two or more analytic regions and one or more non-analytic regions. Each of the analytic regions is provided as a sample interaction region and/or a detection region for the one or more analytes. The two or more analytic regions and the one or more non-analytic regions are provided in one or more substrates in one or more planes; at least one of the two or more analytic regions is formed by a porous material made from particles and a binder; and the two or more analytic regions being separated from the one or more non-analytic regions by diffusion barriers for fluid in the one or more analytic regions. An analysis arrangement, a method of producing a corresponding analysis device, and a corresponding analysis method are also disclosed.

The present invention relates to an analysis device for analysing a liquid sample, a corresponding analysis arrangement, a method of producing a corresponding analysis device, and a corresponding analysis method.

### BACKGROUND

Thin layer chromatography (TLC) is a physicochemical separation method used to investigate the composition of samples of various origins. For example, thin layer chromatography methods are used for routine substance purity or confirmation tests for pharmaceutical drugs, but also as research tools in biology, chemistry and medicine. Particular advantages of thin layer chromatography are low equipment requirements, speed, high separation efficiency and low substance usage.

In thin layer chromatography, a thin layer of small particles (e.g. silica gel, diatomaceous earth, aluminium oxide or cellulose) is typically used as the stationary phase. The stationary phase is uniformly applied to a carrier film or plate produced from materials such as plastic, aluminium or glass. Thin layer chromatography plates are commercially available in different layer thicknesses. Silica gel is usually used as the stationary phase in normal phase thin layer chromatography and serves as a polar adsorbent for the sample molecules due to the free terminal hydroxyl groups. Silica gel or other materials may also be modified, e.g. by hydrophobic groups, or mixed with other materials, such as ion exchange resins, to provide different separation characteristics, e.g. as in reverse-phase thin layer chromatography or ion exchange thin layer chromatography. The present invention is not limited to a specific stationary phase nor to specific separation principles.

Samples for thin layer chromatography often require extensive sample preparation, e.g. to enrich or concentrate components to be analysed or to remove unwanted components (so-called matrix). Furthermore, it is generally desired to perform sample detection directly on the chromatographic plate, in order to reduce the processing effort by e.g. scraping off, extracting and externally analysing chromatographic spots. There is a need for further improvements in thin layer chromatography and other sample treatment techniques.

### SUMMARY

In view of the above, the present invention provides an analysis device for analysing a liquid sample, a corresponding analysis arrangement, a method of producing a corresponding analysis device, and a corresponding analysis method including the features of the independent claims. Preferred embodiments are the subject of the dependent claims and of the description that follows.

An analysis device for analysing a liquid sample containing one or more analytes by thin layer chromatography is proposed, wherein the device comprises two or more analytic regions and one or more non-analytic regions as further explained below.

Each of the analytic regions is provided as a sample interaction region and/or a detection region for the one or more analytes. As to the meaning of these terms, reference is made to the detailed description below. The present invention allows, by providing such a combination of regions, performing multiple stages of an analysis on a single device which may be provided very flexibly and with reduced production effort as compared to known analysis devices. Terms such as “flexibly” or “flexibility” are used herein to indicate that certain devices or methods may be easily configurable or adaptable to specific analysis tasks, analytes, detection methods, etc.

The present invention relates, in particular, to planar separation techniques, i.e., the analytic regions may be provided in one or more planes in any arrangement, particularly in a single plane or two or more parallel planes, as further explained below, providing an improved separation efficiency and flexibility as well as the ability to provide several orthogonal or multi-dimensional (in terms of separation technology) separation and detection for a plurality of analytes. For example, the analytic regions and the non-analytic region may be provided on the same side of one substrate, or on different substrates wherein the analytic regions may face towards each other or be arranged otherwise.

At least one of the two or more analytic regions is formed by a porous material made from particles and a binder, such as indicated below. The present embodiment, at least in aspects, therefore relates to non-paper chromatography, providing an increased flexibility in material selection and allowing for the use of advanced and more flexible techniques for the formation of analytic regions.

The two or more analytic regions are separated from the one or more non-analytic regions by diffusion barriers for fluid in the one or more analytic regions, such that any sample processing is limited to the analytic regions while the non-analytic regions are kept free from components of a sample. By allowing for a large variety of embodiments, the present invention also provides an improved flexibility of preparing a sample analysis device, as further explained below.

In embodiments, each of the diffusion barriers may be provided as a barrier made from a hydrophobic, non-porous and/or non-polar material or being a boundary between the porous material and a region being void of the porous material. That is, the term “diffusion barrier” is generally used herein to describe the edge or boundary between the porous material, in which a liquid may elute, propagate or advance by capillary effects, and a region wherein this is not possible, either because of the porous material being absent or a non-porous or hydrophobic material being present. For specific examples, reference is made to the explanations below. Advantages of providing diffusion barriers accordingly include, again, an improved flexibility and adaptation to manufacturing needs, the samples intended to be treated, and the production methods available.

Providing the analytic regions includes, in an embodiment of the present invention, printing, e.g., screen printing, at least one of the analytic regions onto a substrate. In alternative embodiments, the analytic regions may also be cut from a sheet of material including the particles, and by applying and preferably affixing the cut material to a substrate such as a glass, plastic or ceramics structure.

For certain applications and in embodiments of the invention, drying the plurality of thin layer regions after the screen printing has been found to be particularly advantageous for improving the bonding strength of the analytic regions to the substrate, which may be glass, a ceramics material, or any other type of material in all embodiments of the present invention. Herein, the term “drying” shall refer to any type of removing liquid content, including “actively” drying by applying heat or a stream of a drying gas such as air, nitrogen or a noble gas, but also “passive” drying processes wherein the liquid is allowed to evaporate simply by exposing a component to be dried to ambient conditions. Subsequent to drying, in embodiments of the present invention, a washing step, particularly under the conditions further elaborated below, may be performed, after which a further drying step of the same or of a different kind may be present. For details, reference is made to the detailed description relating to embodiments of the present invention below.

As found by the present inventors, printing, and particularly screen printing, thin layer or analytic regions significantly improves flexibility in providing such regions in virtually any shape and with any properties conceivable. Using a method according to embodiments of the present invention, multifunctional thin layer chromatography devices can be provided wherein the thin layer regions may be provided with different features or characteristics, as defined by the particles used and parameters such as thickness, length, width, shape, and chemical modification. Using screen printing, such regions may be provided with optimized parameters, not being bound by limitations of a conventional coating process. However, screen printing is just one example of a printing process usable according to the present invention, and production processes other than printing may likewise be used according to embodiments, such as, but not limited to, cutting certain shapes from a sheet like material and any other particle deposition process.

Particularly, embodiments of the present invention may include that at least one of the analytic regions, or several analytic regions together, or any combination of one or more analytic regions and one or more supporting regions which is or are, or may be, connected to the analytic regions, comprises a concave shape. The term “concave” is used herein as generally understood in geometry. An alternative term for “concave” is “non-convex”. In a convex shape, any line directly joining two points x and y within the shape lies completely within the shape. The boundary of a convex shape is called a convex curve. A non-convex or concave shape is a shape where the above condition is not met, and a boundary of a concave shape is referred to as concave curve herein. Generally, also a vertical projection of several regions, including analytic and supporting regions, to a plane parallel to these regions may comprise a concave shape. That is, also several regions on different layers or in different planes may, when reduced to a single plane have a concave shape which then may be defined from the regions at different height levels (z-axis). Such regions may, together, also be at least in part enclosed by a diffusion barrier which, at least in a section, has a concave shape, as also further defined below in connection with the detailed description. Such “complex” shapes may particularly advantageously be provided with the techniques described herein.

At least one of the analytic regions may be provided in an elongate shape, and if two or more analytic regions are provided in elongated shapes, their longitudinal axes may be oriented in an acute, right, or obtuse angle to each other. This allows for a two-dimensional separation, for example, where a plurality of analytes may be separated in a first dimension in a first one of the analytic regions and an incompletely separated group of these analytes may then be separated, using the same or different separation principles, in a second dimension in a second one of the analytic regions.

In embodiments of the present invention, at least one of the analytic regions may also be completely enclosed by at least one of the diffusion barriers. In such a configuration, two of such analytic regions may be “bridged” using a bridging element which is placed in contact with the analytic regions, such that fluid can pass between such regions. This may particularly help in clearly defining the point of time when a separation, for example, shall start, and experimental conditions may be more clearly defined.

In embodiments of the present invention, the particles are provided to interact with the analyte or at least one of the analytes or any other sample component as explained below by at least one of noncovalent interactions selected from polar, hydrophobic, ionic and interactions on the basis of biorecognition, such as immunogenic interactions or more generally interactions including antibodies, aptamers, etc., and covalent interactions. Combinations of particles in the same analytic region or in different analytic regions may be provided, for example, to enable an analyte enrichment in one of the regions, to effectively separate the sample in a further one of the regions, and to perform a selective detection in a yet further one of the regions.

The device according to the present invention may also comprise further regions termed “supporting regions” herein and may therefore, in embodiments of the present invention, be provided to include one or more sample application regions, mobile phase application regions, bridging and interconnection regions, additionally to the analytic regions which may e.g. comprise sample or analyte separating regions, sample interaction regions, sample or analyte detection regions, sample or analyte enrichment or solid phase extraction regions. Herein, the terms “sample” and “analyte” are synonymously used in combination with terms such as “separation”, “detection” and “enrichment”, noting that a certain separation, detection and enrichment technique may generally affect all, or more preferably a subset of, components of the sample, and particularly and intentionally only specific analytes, which are components of the sample for which an analysis is intended. The interconnection regions may particularly be provided for connecting other regions including the analytic with each other or with the supporting regions. A bridging region may be provided in a different plane and may be brought into contact with regions to be connected. As to the meaning of these terms, reference is made to the explanations relating to specific embodiments below. Particularly when providing combinations of such regions with different properties, an analysis device provided by a method according to corresponding embodiments of the present invention is an integrated and multifunctional device obviating the need for separate sample concentration and detection processes, for example, and reducing analysis time and effort as well as the likelihood of errors.

In embodiments of the present invention, a plurality of the interconnection regions may be provided, i.e. further regions which may be provided to connect at least two further ones of the regions mentioned in the form of parallel connections. This allows for restricting or shaping a liquid flow between the connected regions. Additionally, or alternatively, at least one of the thin layer regions may be provided to connect to at least a further one of the thin layer regions and/or a further region, such as a supporting region or an interconnecting region, via one or more tapered transitions, which either widen or narrow in the direction of a liquid flow. In the former alternative, the liquid may be distributed across the whole breadth of a downstream region, and in the latter alternative, liquid may be focussed to a small spot or a specific region, e.g. a thin layer region in which an enrichment or detection may be performed. In the latter configuration, the tapering region has a function resembling that of a funnel.

Yet additionally, or alternatively, at least one of any of the regions referred to above may be provided in a longitudinal and bent shape to correspond to an area or shape of the substrate. In such an embodiment, a relatively long separation path, for example, may be provided on a relatively small substrate. Further modifications are possible in this connection, e.g. providing an additional side channel or slanted border of the thin layer region under consideration in the region of the bent, in order to improve fluid flow characteristics therein.

Yet additionally, or alternatively, at least one of the thin layer regions may be provided to have a greater layer thickness as compared to at least a further one of the thin layer regions, e.g. by double or multiple screen printing. A thin layer region provided accordingly may be used to receive a relatively large amount of liquid phase or eluent, or it may, in case functionalized particles are used in such a region, it may be provided as a solid phase extraction region to which sample is applied. In such a configuration, the amount of analyte bondable to the functionalized particles in this region is increased by providing it in a greater thickness.

Partly repeating what was already stated above, at least two of the thin layer regions may also be provided as disjoint thin layer regions separated by a region of a substrate kept void of particles. Such regions may be, as further explained below, be temporarily interconnected by a liquid bridge or bridging structure, e.g. an element comprising a further thin layer region which is placed in contact with both separated regions. In this configuration, selective valve functions may be provided on a device according to an embodiment of the present invention.

At least two of the regions may, in further alternative or additional configurations, and as also mentioned, be provided to have longitudinal shapes with maximum extensions which are oriented in an angle to each other on a substrate. In such a configuration, a sample may first be separated in a first direction, e.g., in a sample separation region, and analytes isolated in the sample separation region may be eluted towards a further region, e.g. for detection, in an elution direction which is oriented in an angle, such as 90°, to the extension of the sample separation region.

In embodiments of the present invention, the particles may be provided as modified or unmodified silica, microcellulose, carbon, and/or polymer based particles, and at least one of the printing fluids, if a screen printing process is used, may be provided to include xanthan gum as a binder. Modifications may particularly include attaching hydrophobic moieties to the silica particles or otherwise functionalizing the same. Xanthan gum was, as further explained below, found to represent a thickener with particularly advantageous properties. For specific conditions and compositions, reference is made to the explanations below.

An analysis device for analysing a liquid sample containing one or more analytes by thin layer chromatography provided herein comprises a planar substrate and a plurality of thin layer regions on the substrate, wherein the thin layer regions are provided by screen printing the thin layer regions using one or more printing fluids, the printing fluid or at least one of the printing fluids comprising particles interacting with the analyte or at least one the analytes.

As to specific further features and advantages of such an analysis device, reference is made to the explanations above relating to preferred embodiments of an inventive method, as these explanations likewise apply to the analysis device provided accordingly. Further reference is made to the explanations below related to specific embodiments of the present invention. Corresponding explanations also apply to an analysis device which is provided according to a method according to any of the embodiments of the present invention explained above.

In such an analysis device, particularly, at least one of the thin layer regions may be provided as a solid phase extraction region, a thin layer chromatography region, a biorecognition region or an immunoassay region or combinations of such functions may be provided. Particularly in combination of such regions, a corresponding analysis device is an integrated means for analyte concentration and/or separation and/or detection. Sample concentration may, in all embodiments of the present invention or a specific subset thereof, be performed to enrich certain components. Sample separation may be performed using all known separation principles including, but not limited to, normal phase or reverse phase separation, ion chromatography or size exclusion chromatography (where an interaction is based on known size exclusion effects), and sample detection may e.g. include colorimetric or immunogenic detection.

Embodiments of the present invention also relate to an analysis arrangement comprising an analysis device according to any one of the embodiments referred to herein and a covering structure, the covering structure being configured to position and to at least partly cover the analysis device. A covering structure may represent a portable cartridge with defined features which, for example, may assist an analyst in applying a sample and/or any separation or detection liquid. A corresponding cartridge may also be used in an automated analysis system, which may be used in an embodiment of the present invention, and which may comprise liquid dispensing means and may also be adapted to provide a readout of a analyte detection reaction, according to certain embodiments.

According to an embodiment of the present invention, the covering structure comprises a receptacle configured to receive a liquid reservoir containing a liquid and piercing means configured to pierce the fluid reservoir upon operating an operating means associated with the receptacle. Operating the operating means may be performed by hand by an analyst or, if used in an automated sample analysis system, by, e.g., mechanical or automated means such as an electromechanical actuator, e.g. a piston depressing a button to which piercing means are attached or otherwise functionally connected. The receptacle is provided such that the liquid which is contained in the liquid reservoir is applied to a defined region of the analysis device when the reservoir is pierced. In such an embodiment, a defined amount of liquid in a controlled composition may always be dispensed to the analysis device, reducing human error and simplifying operation. By providing a specific amount of liquid in an enclosed space, furthermore, such as in the liquid reservoir, degradation or spoilage effects may be minimized in corresponding embodiments.

In embodiments of the present invention, the covering structure may, alternatively or additionally, comprise at least one cutout for applying the sample to the analysis device and/or at least one cutout for applying a further liquid to the analysis device, such as to rinse a sample preconcentration region, for example. Additionally or alternatively, cutouts may be present for providing access to an analyte detection region, such as to be able to assess a detection reaction, either by a human user or by an analysis system, and/or controlling air exchange, in order to prevent or enhance drying. Providing specific cutouts also simplifies operation as only access to one or more defined positions on the analysis device may be provided, and dispensing means such as a capillary or pipette may be guided to such a position. Cutouts provided in embodiments of the present invention may also comprise closing means, such as shutter sliders, e.g. to avoid unwanted exposure to light, e.g. for optically sensitive samples, or drying.

Aspects of the present invention also relate to a method for producing an analysis device for analysing a liquid sample containing one or more analytes, wherein the method comprises the steps of providing one or more planar substrates and providing at least one analytic regions on the one or more substrates. Said providing the one or more analytic regions comprises applying a porous material made from particles and a binder onto the one or more substrates. As to further features and advantages of a corresponding method, which are evident from the device features referred to before, reference is made to the corresponding explanations above.

In such a method, said providing the one or more analytic regions made from particles and a binder may particularly comprise screen printing a slurry comprising said particles and said binder onto the substrate. This also applies for a method of analysing a liquid sample containing one or more analytes, wherein an analysis device as described above is used.

A method of analysing a liquid sample containing one or more analytes, particularly by thin layer chromatography, is also part of the present invention. The method comprises providing an analysis device or an analysis arrangement as explained in embodiments before, and therefore likewise takes profit of the advantages of the features thereof. Reference is therefore made to the explanations above.

The method may comprise applying or transferring the sample to at least one of the analytic regions, and at least one of separating components of the sample, enriching the analyte or at least one of the analytes, collecting the analyte or at least one of the analytes, reacting the analyte or at least one of the analytes, binding the analyte or at least one of the analytes, and detecting the analyte or at least one of the analytes in at least one of the analytic regions or performing a transfer between such regions. Particularly combinations of such sample or analyte treatment steps are suitable for an integrated and user friendly analysis.

The present invention may be used with virtually all samples suitable for separation in separation methods such as thin layer chromatography or other separation techniques. The sample may be selected, for example, from at least one of a medical sample, a therapeutic drug, an environmental sample, a food sample, a water sample, a forensic sample, an animal feed sample, and a sample produced in an industrial process, e.g. in connection with production process monitoring.

Likewise, all analytes separable by thin layer chromatography may be used, wherein the analyte or at least one of the analytes may particularly be selected from at least one of a vitamin, a hormone, a biomarker including at least one of a carnitine, an amino acid, a fatty acid and a neurotransmitter, a pesticide, an organic contaminant including at least one of a therapeutic drug, a hormone and a perchloro or perfluoro compound, a mycotoxin, a compound known as an allergen, a polyaromatic hydrocarbon, acrylamide, an adulterant, melamine, an additive including at least one of a preservative, a food colorant and a sweetener, a drugs of abuse, an explosive, an algal toxin, a doping compound, a nutrient, a diagnostic substance, and a natural compound. The present invention is not limited in any way by either the separation technology or the type of sample.

### SHORT DESCRIPTION OF THE FIGURES

Embodiments of the invention will now be described, by way of example only, with reference to accompanying drawings, in which

FIG. 1 illustrates a device according to an embodiment of the invention;

FIG. 2 illustrates an analysis using a device according to FIG. 1;

FIG. 3 illustrates a device according to a further embodiment of the invention;

FIG. 4 illustrates an analysis using a device according to FIG. 3;

FIG. 5 illustrates a device according to a further embodiment of the invention;

FIGS. 6A to 6E illustrate steps of an analysis using a device according to FIG. 5;

FIGS. 7A to 7D illustrate aspects of a device according to FIG. 5;

FIG. 8 illustrates an arrangement according to an embodiment of the invention;

FIG. 9 a method according to an embodiment of the invention.

### DETAILED DESCRIPTION

In the Figures, elements of identical, essentially identical, functionally comparable, or technically compatible function and/or purpose may be identified with identical reference numerals, and repeated explanations may be omitted for reasons of conciseness. Explanations herein relating to devices, apparatus, arrangements, systems, etc., according to embodiments of the present invention likewise may apply to methods, processes, procedures, etc. according to embodiments of the present invention and vice versa.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future, particularly when encompassed by the scope of the independent claims.

Specific embodiments of the present invention may relate to the analysis of biotin as an analyte, particularly in a biological sample, using a sample analysis device prepared including a screen printing process, but the present invention is not limited to this type of sample, analyte, and production process for an analytical device, but only by the scope of the independent claims.

Before again turning to features and advantages of embodiments of the present invention, some underlying information as to the understanding and extent of some terms used herein as well as to biotin analysis and screen printing processes will be given, in order to better understand the features and advantages of the embodiments of the present invention and embodiments thereof.

Generally, the term “analyte” shall refer, in the understanding underlying the present invention, to any component which is intended to be detected, analysed or measured in a corresponding method, such as a molecule, an aggregate of molecules, an ion, or an aggregate of ions, that may be separated from other components by techniques such as thin layer chromatography, particularly by making such a component to interact with one or more of the analyte interaction regions as provided according to embodiments of the present invention or the particles contained in such sample interaction regions.

A “sample”, in the understanding used herein, may contain one or more of such analytes, particularly in a solubilized state, and one or more solvents selected from one or more organic or inorganic solvents, polar or apolar solvents and protic or aprotic solvents, such as water, alcohols, and other organic solvents. An analysis method may particularly have the purpose of whether or not a sample contains the analyte, and in which concentration.

One or more modifiers may be added as well, such as one or more acids, one or more alkaline compounds, or one or more buffering substances, particularly to influence the pH value. A sample may be provided, e.g., as a solution, a suspension, a colloid, and an emulsion, and the present invention is not limited to artificial samples where analytes are dissolved or reconstituted in a laboratory by addition of solvent(s). Embodiments of the present invention may also relate to natural samples, such as body fluids, e.g., full blood, blood plasma, urine, sweat, saliva, lymphatic fluid, cerebrospinal fluid, etc., whether or not provided in an untreated or treated form or treated by pre-processing methods such as filtration, centrifugation, sedimentation, cell disruption, drying, heating, cooling, addition of solvent(s) and any other method. The term “solvent” is intended to include an added solvent and a natural solvent being part of the sample in its original form. Herein, if reference is made to a “sample” or “analyte” in the singular, a plurality of samples or analytes shall likewise be covered by analogy.

Herein, the term “sample interaction region” or “analyte interaction region” is intended to refer to any type of analytic region intended and configured to interaction with an analytic sample or components thereof, particularly by providing a stronger retention in view or certain sample components as compared to others, “sample components” referring to one or more analytes to be detected or detectable, so-called “matrix”, i.e. components other than the analytes present in a sample, irrespective of whether interfering with an analysis, separation and detection of the analyte(s) or not, and an, e.g., aqueous or non-aqueous (e.g., organic) liquid solvent or mixture in which the sample components are dissolved, reconstituted, dispersed, or otherwise provided to be analysed. As mentioned, “sample” and “analyte” may be used synonymously in some contexts. The term “interaction” may refer to mechanisms resulting in a separation, but also resulting in a (pre)concentration of sample components. In the former, different analytes typically may interact to different degrees, in any manner conceivable, with a stationary phase, such that they are separated in a separation process. In the latter, one or more analytes of a larger sample volume may be retained on a stationary phase while further components not or less interacting with the stationary phase, including in particular matrix compounds and solvents, may be removed, such as in solid phase extraction.

As mentioned, the present invention includes providing diffusion barriers between analytic and non-analytic regions, and each of the diffusion barriers may be provided as a barrier made from a hydrophobic, non-porous and/or non-polar material or being a boundary between the porous material and a region being void of the porous material. The term “diffusion barrier”, therefore, may relate to any form of boundary, border or delimitation for regions which block diffusion of liquid, or liquid of a specific chemical characteristic, e.g., an aqueous or organic liquid or a certain mixture. For example, an analytic region in the form of, or comprising a, liquid-diffusible porous layer may be separated from a likewise or similarly liquid-diffusible non-analytic region by a diffusion barrier formed of a solid, hydrophobic or hydrophobized line or strip forming the diffusion barrier. In such an embodiment, the diffusion barrier may be, e.g., produced by applying wax, grease or oil of any type, particularly of a material not interacting, other by blocking diffusion, with any component of the sample. In other embodiments, an analytic region is liquid-diffusible while a non-analytic region may generally be less or essentially not liquid-diffusible, either by providing the non-analytic region as a region void of porous material or made from a different material. When, e.g., using printing techniques, the analytic regions may be printed onto a substrate while leaving the non-analytic regions void, therefore realizing a diffusion barrier according to the understanding as used herein. When covering a larger part of a substrate with a porous material, the analytic and non-analytic regions may also be separated from each other by scraping off, or otherwise removing, the porous material in a strip, trench or line, or in a larger portion of the substrate, such as to form the diffusion barrier.

As mentioned before, embodiments of the invention may include that at least one of the analytic regions, or several analytic regions in common, or at least one analytic region and at least one supporting region are provided in a concave shape or enclosed by a diffusion barrier which has a concave shape, at least in a section. In this connection, the term “concave” shall particularly indicate that the region(s) referred to accordingly comprise(s) an indentation formed by straight or (regularly or irregular) bent line. As mentioned, this may also be the case for regions in several planes when virtually reduced to a single plane, i.e., in a vertical projection. That is, the analytic regions, or several analytic regions in common, or a corresponding projection of several regions in different planes, may include a first straight border line corresponding or not corresponding to a diffusion barrier, and a second straight border line corresponding or not corresponding to a diffusion barrier, wherein the first and second border line enclose an interior angle between of more than 180 degrees, e.g., 270 degrees.

The “interior angle” shall be the angle enclosed by the sides of the border lines facing towards the analytic regions(s). In such a configuration, and also for non-straight border lines, such as regularly or irregularly curved border lines, a straight line can be drawn between two points, each of which being within the interior of the analytic region(s) that dissects the border line(s) twice. Generally, a contiguous border line of one or several analytic regions has at least five, six, seven, or eight angles, in certain embodiments of the present invention.

Biotin, also known as vitamin B7 or H, is a coenzyme involved in multiple metabolic processes, including carbohydrate metabolism, fatty acid synthesis, amino acid catabolism, and gluconeogenesis, and is essential for DNA synthesis and cell replication as well. In nature, biotin is found in high concentrations in egg yolks, soybeans, liver, and yeast, as well as a variety of plants, albeit at lower levels. On the market, biotin is often found in multi-vitamin preparations, prenatal vitamin preparations, and dietary supplements marketed for improving hair, skin, and nail growth or condition. Recommended intake of biotin for adults is in the range of 30 to 75 μg/day. Beneficial effects of high-dose biotin have been demonstrated in progressive multiple sclerosis (MS).

However, nowadays supraphysiological doses of biotin (up to 30 mg/day) are widely used for self-medication aimed at reducing hair loss or improving nail or skin condition. Unfortunately, these supraphysiologic biotin intakes have produced emerging problems with immunoassays that utilize the streptavidin-biotin interaction. Because the high circulating biotin concentrations produce positive or negative interference in biotinylated competitive and noncompetitive (sandwich) immunoassays, respectively, and these types of immunoassays account for about half of all used current immunoassays. Case reports and in vivo studies show that ingestion of supplemental biotin can cause clinically significant errors in a significant amount of biotinylated immunoassays. The misdiagnosis and mismanagement of patients have been reported after thyroid-function tests due to the biotin interference. According to the U.S. Food and Drug Administration (FDA), a patient with a high intake of supplemental biotin died following a troponin test (to help diagnose a heart attack) that gave a falsely low result because the test was subject to biotin interference. In its 2017 Safety Communication, the FDA issued recommendations to consumers, healthcare providers, laboratory personnel, and lab-test manufacturers regarding biotin interference.

This issue of biotin interference is particularly critical for patients in emergency situations as a quick test for biotin is still not available to confirm whether the patient has been subjected to high-dose biotin. At the moment, in these types of time critical situations, rapid screening of biotin is highly needed to provide a warning to possible falsely negative or falsely positive outcomes of biotinylated immunoassays before the sample is subject to the immunoassays.

An overview about existing analysis methods for measuring biotin may be found in several literature reviews. The present invention contributes to these and particularly allows for a rapid, reliable and user friendly biotin analysis at least partially overcoming the disadvantages mentioned before. While biotin analysis is a particularly advantageous field of application of the present invention, however, the present invention and its embodiments may be used in a wide field of applications, as mentioned, both in terms of samples and analytes.

The present inventors have found that a set of new methods recently created by them to fabricate microfluidic systems are particularly suited for thin layer chromatography analysis and related analyte concentration and analyte detection systems. Reference is particularly made to an article by H. Evard et al., “A New Direction in Microfluidics: Printed Porous Materials”, Micromachines 2021, 12(6), 671, DOI: 10.3390/mi12060671, the content of which is incorporated herein by reference.

Comparable to microfluidic paper-based analytical devices, in these methods porous materials were used. However, alternative porous materials and different printing methods were used here to give the material the necessary pattern to act as a microfluidic system. While, microfluidic systems were produced by (1) curing a porous monolithic polymer sheet into a necessary pattern with photolithography, (2) screen printing silica gel particles with gypsum, and (3) dispensing silica gel particles with polyvinyl acetate binder using a modified 3D printer, screen printing was found to be a particularly advantageous method for providing a patterned layer of porous material.

Screen printing is a printing process in which the printing ink is forced through a fine-mesh fabric onto the material to be printed using a rubber squeegee. At those points of the fabric where no ink is to be printed according to the print image, the mesh openings of the fabric are made impermeable to ink by means of a stencil.

In the screen printing process, it is possible to print on many different materials, both flat (foils, plates, etc.) and shaped (bottles, equipment housings, etc.). Special printing inks are used for this purpose, depending on the material. Mainly paper products, plastics, textiles, ceramics, metal, wood and glass are used as printing substrate. The print format ranges, depending on the application, from a few centimeters to several meters. One advantage of screen printing is that the ink application can be varied by using different mesh count, so that high ink layer thicknesses can be achieved. Screen printing is mainly used in advertising and lettering, textile and ceramic printing, and for industrial applications. Any screen printing process, such as disclosed in textbooks like MacDougall, A., “Screen Printing Today: The Basics”, Media Group, 2008, may be used.

In embodiments of the present invention, a printing fluid or “ink” may be prepared from an aqueous solution of xanthan gum with concentration of 0.2 to 2%, particularly 0.4% and adding a weighted amount of particles to get a specific particle concentration such as 300 to 600 mg per millilitre of the solution. Such values can change significantly when the manufacturer of a batch of reagent is different, or even from batch to batch, and therefore will be selected by the skilled person depending on the particular circumstances. A suitable amount of glycerol may be added to the mixture for getting a desired glycerol concentration, e.g. 50 to 100, particularly 75, microlitres of glycerol per millilitre of xanthan gum solution. The silica gel particles particularly are, in an embodiment, unmodified silica gel particles provided in a size distribution of 10 to 20 μm, e.g. a mean size of 15 μm. Using xanthan gum in a corresponding process is particularly advantageous as this was found to be a particularly suitable thickener and binder which holds particulate material together and attaches the same to the substrate. At different concentrations than those indicated, less advantageous results in terms of screen printing results, attachment to the substrate, etc., were observed.

As substrates, generally all substrates may be considered in embodiments of the present invention. However, glass was found to be of particular advantage, such as compared to aluminium, plastics such as polyethylene terephthalate (PET), polyethylene (PE), etc., as printing on glass resulted in less detachment (flaking) which may, without being bound by theory, be the result of covalent interactions between the glass surface and the xanthan gum.

In processes according to embodiments of the present invention, a post screen printing treating sequence including drying and washing was found particularly advantageous. Drying may be performed in an oven at a temperature of particularly 60 to 180 °C, while this temperature is not specifically limited as long as no degradation of either of the components is observed, for a sufficient time of, e.g., 0.1 to 12 hours, particularly about 2 hours. The washing step may include soaking in water for a certain time of, e.g., 0.1 to 5 hours, particularly about 1 hour. Drying was found to be particularly advantageous to make the printed material stronger, while washing was found to be particularly advantageous to remove excess xanthan gum and glycerol which otherwise could interfere with later analysis. Printing may also be performed without glycerol and the degree of removal of xanthan gum or any loose material or compound may vary.

Aspects of the present invention relate to solid phase extraction and detection by immunoassays and colorimetric detection.

Solid phase extraction (SPE) is a sample preparation method used for enrichment, concentration and/or isolation of an analyte. It is based on a physical extraction process between a liquid and a solid phase, also called sorbent, similar to column chromatography. However, the term is to be understood broader and encompasses a whole range of sorbent/analyte interactions: polar and nonpolar interactions, ionic interactions, interactions via covalent bonds, and any combination of such interactions.

An analyte provided in a liquid sample may, in solid phase extraction, be enriched from very dilute solutions for an analysis. While sample solution is passed through the sorbent, the analyte accumulates on the sorbent bed, with the solvent passing through the sorbent with less or no interaction. The choice of sorbent has a great influence on the extraction. A suitable solvent is thereafter used to elute the analyte from the sorbent. An eluate thus formed can be further processed or analysed with a suitable analysis method. Solid phase extraction is generally considered to be a fast and inexpensive analytical method, which is suitable for large sample quantities and a high degree of automation.

Immunoassays are methods in bioanalysis whose common basic principle is the detection of an analyte in a liquid phase by binding an antigen to an antibody. Depending on the configuration of the assay, both antigen and antibody may be the analyte to be detected. Immunoassays take advantage of the high specificity and binding strength of binding between antigens and antibodies. In immunoassays, the analyte is present in a liquid phase, usually dissolved in a buffer. Immunoassays are based on using labelled reagents for detection and quantitative determination. Depending on the assay format, application and desired sensitivity, different labelling techniques are possible.

Widely used is labelling using enzymes that catalyse a chemical reaction in which either a reaction substrate produces a specific colour (chromogenic substrate) or light is emitted via luminescence. Another optical method is labelling with fluorescent dyes. Both the colour intensity for chromogenic substrates and the light intensity for luminescent and fluorescent substrates or labels, respectively, can be measured with appropriate instruments. Other options for labelling include binding the detection reagent to gold colloids or to coloured polymer particles. In both cases, visual detection with the naked eye is possible by binding a sufficient amount. Less commonly used are labelling with magnetic particles and thus measurement of magnetic field strength as the detection principle, and electrochemical methods, for example based on impedance measurements.

Embodiments of the present invention may furthermore be used in connection with more conventional colorimetric analysis techniques, i.e. methods of determining the presence and/or concentration of a chemical element or chemical compound in a solution with the aid of a colour reagent. Colorimetric detection is applicable to both organic compounds and inorganic compounds and may be used with or without an enzymatic stage. In embodiments of the present invention, colorimetric detection may be used in connection with metal cation analysis and is explained below as an embodiment of the present invention.

In embodiments of the present invention, layers of porous particles may be provided on a substrate in any shape and varying thickness and modification, forming analytic regions as repeatedly mentioned before, such that various combinations of different analysis principles on a single substrate, or several substrates in a single device, are possible, as will now be explained with reference to different embodiments of the present invention.

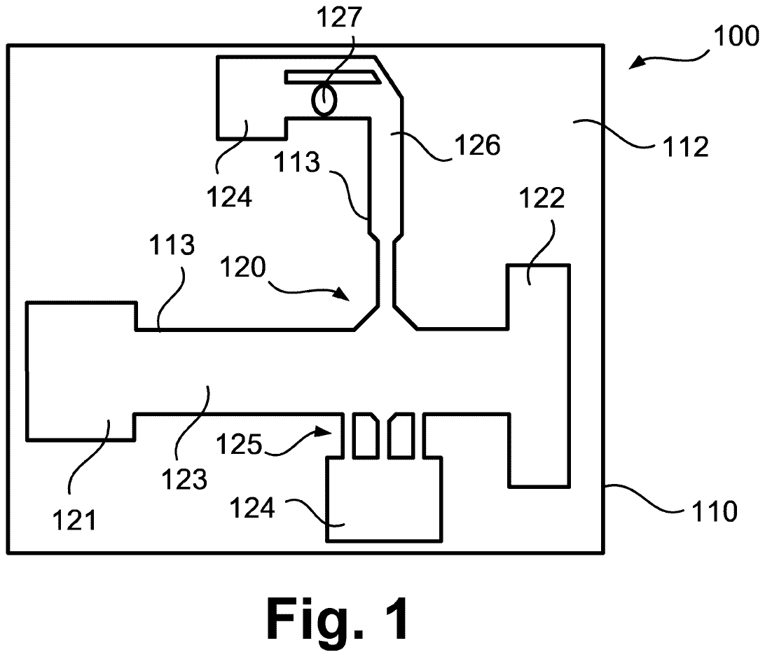

In this connection, FIG. 1 schematically illustrates a device according to an embodiment of the present invention which is designated 100. The device comprises a substrate 110, particularly a planar substrate such as a glass plate or a plate of any other suitable material. On the substrate, a pattern of thin-layer material is screen printed or applied to using any other method conceivable, said pattern being referred to with 120 as a whole.

The pattern 120 is, in the example illustrated, formed to include a mobile phase application region 121, a mobile phase sink or waste region 122, a sample or analyte separation or thin layer chromatography region 123 as an analytic region, eluent application regions 124, interconnection regions 125, a sample interaction region 126 as a further analytic region, and a detection substance application region 127, wherein the mobile phase application region 121 serves the purpose of applying a mobile phase, either by hand or in an automated system, and the mobile phase sink or waste region 122 serves the purpose of allowing a larger amount of mobile phase to pass through the sample separation or thin layer chromatography region 123 in which components of the sample, including an analyte, may be separated. The purpose and function of the further regions, i.e. the interconnection regions 125, the sample interaction region 126, and the detection substance application region 127 will be evident from the following explanations relating to the function or operation of the device 100 in an embodiment of the invention. At least some of the regions, including the thin layer chromatography region 123 and the sample interaction region 126 include particles as explained above.

The ensemble of thin layer regions, including the analytic regions mentioned, is surrounded by a space of the substrate 110 void of the particles, which is referred to one or more non-analytic regions 112 herein. Between the analytic regions, such as the thin layer chromatography region 123 and the sample interaction region 126 and the non-analytic region(s) 112 adjacent thereto, diffusion barriers 113 are provided as edges or transition lines between the porous material of the analytic regions, supporting regions 122, etc., and the non-analytic region(s). Corresponding diffusion barriers 113 are provided between the other regions and the non-analytic region(s) as well in the example shown.

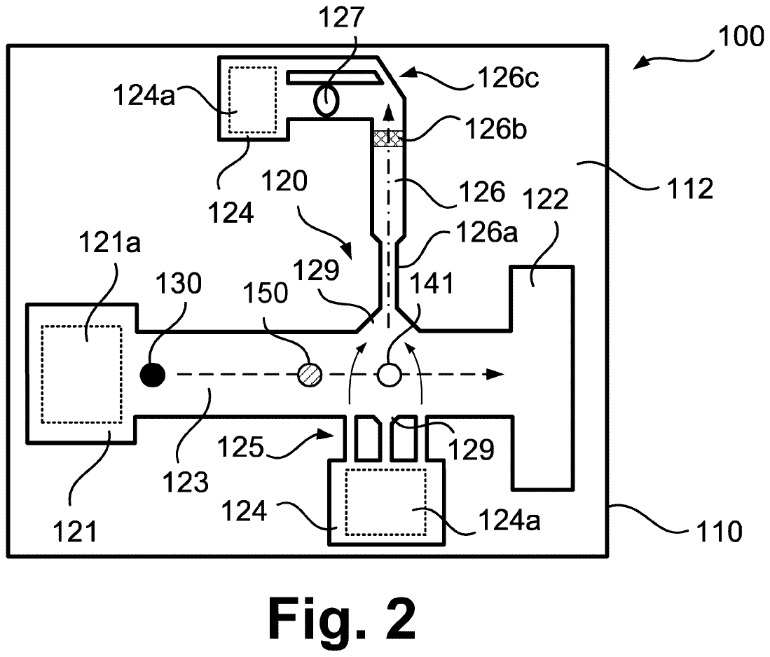

In FIG. 2, an analysis using a device according to FIG. 1 is schematically illustrated, wherein the same reference numerals as in FIG. 1 are used in part and reference numerals for the diffusion barriers 113 are omitted for reasons of clarity only. The analysis is illustrated for biotin but, as mentioned not limited thereto. The shapes and arrangements of the regions 121 to 127 may be varied as needed, e.g. for analysing other and further analytes. Depending on separation and detection principles used in a correspondingly modified device, certain regions 121 to 127 may be omitted or further regions can be added.

As illustrated in FIG. 2, the mobile phase application region 121 may comprise a subregion 121a which is shown with a dotted line and which may be provided with a larger thickness than the remainder of mobile phase application region 121, in order to be able to receive a larger amount of mobile phase. Subregion 121a may be formed by double screen printing or multiple screen printing or by using any technique suitable for creating a greater layer thickness. As further explained below, device 100 may be used with a liquid reservoir in a cartridge or covering structure 500 and is therefore advantageously provided with a larger area than a liquid outlet of the cartridge. By increasing the thickness of the layer in subregion 121, emptying a liquid reservoir is improved and unwanted liquid flows are avoided.

At a position at the start of the sample separation region 123, a liquid sample 130 illustrated with a solid spot is applied (“spotted”) onto the sample separation region 123 and may be dried thereafter before a suitable mobile phase is applied in mobile phase application region 121. Under the influence of the mobile phase propagating or eluting along sample separation region 123 from the mobile phase application region 121 to the mobile phase sink region 122, as illustrated with a dashed arrow, the sample 130 is separated along the sample separation 123 such that an analyte 141, which may be biotin in the example illustrated, may be separated from further analytes or sample components 150 like background or matrix compounds.

After a sufficient separation has been achieved, the mobile phase may be actively or passively dried and an eluent may be applied to an eluent application region 124 shown in the lower part of FIGS. 1 and 2 which may, as the mobile phase application region, be provided with a thicker subregion 124 a. For subregion 124a, essentially the same explanations apply as for subregion 121a of the mobile phase application region 121. A suitable eluent may propagate from the eluent application region 124 shown in the lower part of FIGS. 1 and 2, or from its subregion 124a, via interconnection regions 125.

In the embodiment illustrated in FIGS. 1 and 2, a plurality of the interconnection regions 125 is provided to connect the eluent application region 124 or subregion 124a and the sample separation region 123 in the form of parallel connections. In relation to this embodiment, and all other embodiments provided according to embodiments of the present invention, the term “parallel” is not necessarily to be understood in a geometrical sense but in the sense of providing multiple paths arranged side by side, such as in “parallel” electrical connections.

Parallel interconnection regions 125 may, in this and all further embodiments of the present invention where they are provided, particularly include three interconnection regions 125 which are dimensioned such that an amount of liquid propagating through these interconnection regions generates a flow at the outlet of the interconnection regions which, as illustrated with solid small arrows, focusses the analyte 141 to a certain position. Furthermore, providing several small interconnection regions 125 instead of one larger interconnection region reduces negative influence on the mobile phase flow in sample separation region 123 in the preceding separation step.

As illustrated in FIGS. 1 and 2, the central one of the interconnection regions 125 opens towards the sample separation region 123 via a tapered transition 129 and the sample separation region connects to an initial region 126a of the analyte interaction region 126 via a further tapered transition 129. The former serves the purpose of somewhat distributing or dispersing the eluent propagating from the central one of the interconnection regions towards the analyte 141, while the latter focusses or funnels the eluent comprising the eluted analyte 141 towards an initial part 126a of the sample interaction region 126. Such dispersion and focusing functions of tapered regions 129 may be utilized according to all embodiments of the present invention where they are present and are not limited to the embodiment shown in FIG. 1.

Elution with eluent provided from eluent application region 124 in the lower part of FIGS. 1 and 2 proceeds in an upward direction (in the drawing) as illustrated with a dash-dotted arrow. Again, the initial region 126a of the analyte interaction region 126 is provided with a smaller dimension, e.g. to reduce an influence on the mobile phase flow in sample separation region 123 and therefore separation of the analyte 141 therein. While passing over a subregion 126b of the analyte interaction region 126 supplied with suitable and immobilized biorecognition element, for example, the analyte 141 is caused to bind in this subregion.

A detection substance is applied to detection substance application region 127 from which it flows against the direction of the dash-dotted arrow and across the subregion 126b with the analyte 141 immobilized to the biorecognition element when eluted with an eluent applied to eluent application region 124 in the upper part of FIGS. 1 and 2, which may also comprise a subregion 124a as explained. The detection substance (in the specific example shown a biotin-fluorescein conjugate) is thus added and dried in region 127. Eluent from region 124 in the upper part of FIGS. 1 and 2 elutes the detection substance to the right and then down over the region 126b. In this region, a visual or instrumental detection may be performed.

The analyte interaction region 126 comprises, as commonly designated 126c, a bend and two parallel strands which may be used to increase a length thereof while fitting onto the substrate 110. The extra channel and the sloped portion in region 126c serve the purpose of better directing the detection substance or detection compound from region 127 over subregion 126b.

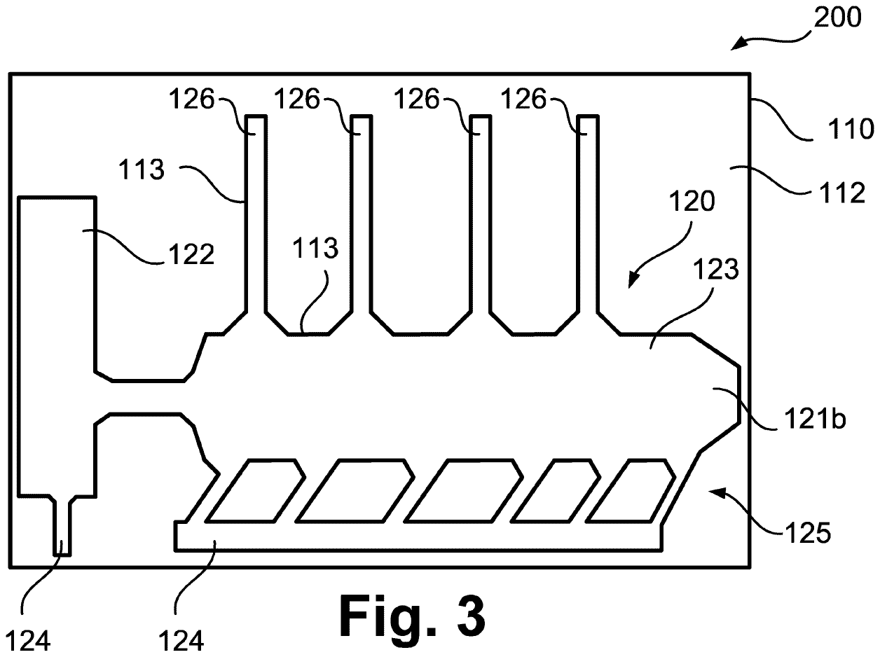

FIG. 3 schematically illustrates a device according to a further embodiment of the present invention which is designated 200. As the device 100 shown in FIGS. 1 and 2, the device 200 comprises a substrate 110. On the substrate, a pattern of thin-layer material is screen printed, said pattern being referred to with 120 as above, and leaving non-analytic region(s). The embodiment of the device 200 shown in FIG. 2 is particularly adapted to perform metal cation detection, but can also be used for other purposes and may be adapted in shape and functions, if necessary. Again, any regions of device 200, and pattern 120 as a whole, may be formed using techniques other than screen printing.

The pattern 120 is, in the example illustrated, again formed to include a mobile phase sink region 122, a sample separation or thin layer chromatography region 123 as an analytic region, eluent application regions 124, interconnection regions 125, and sample interaction regions 126 as further analytic regions. As to the functions of these regions, reference is made to the explanations above. At a position indicated with 121b, the device 200 may be placed into a thin layer chromatography tank with mobile phase, but a mobile phase application region 121, such as illustrated in FIGS. 1 and 2, may be provided in an alternative. Diffusion barriers 113 are provided as explained before.

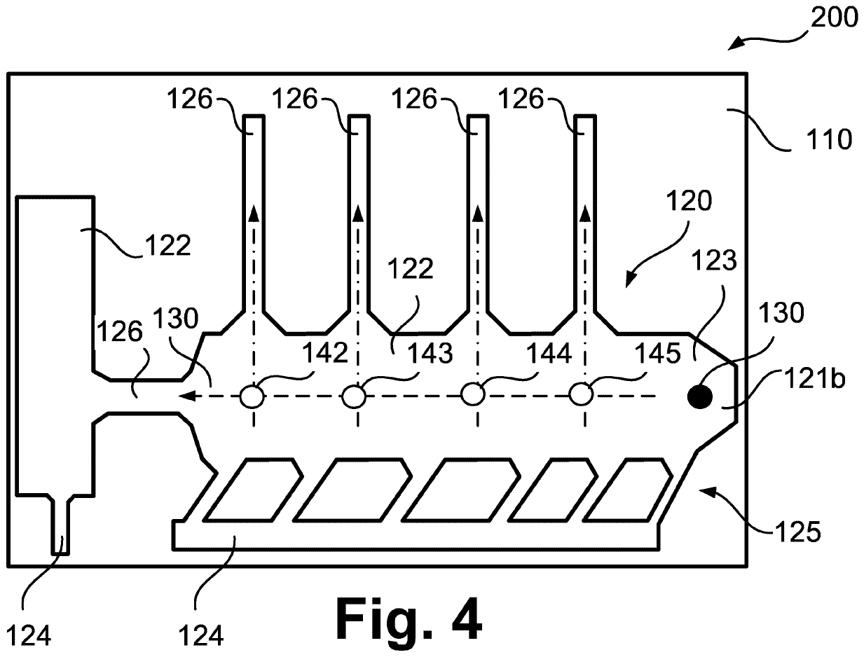

In FIG. 4, an analysis using a device 200 according to FIG. 3 is schematically illustrated, wherein the same reference numerals as in FIG. 3 are used.

Essentially as explained in connection with FIG. 2, at a position at the start of the sample separation region 123, a liquid sample 130 illustrated with a solid spot is applied (“spotted”) and may be dried thereafter before a suitable mobile phase is applied by placing region 121b into the mobile phase in the thin layer chromatography tank. Under the influence of the mobile phase propagating along sample separation region 123, as illustrated with a dashed arrow, the sample 130 is separated along the sample separation region 123 such that analytes 142 to 145, which may be metal cations in the example illustrated, may be separated from each other and/or from other sample components. An analyte, such as nickel cations, may also be eluted directly to an analyte interaction region 126 illustrated leftmost in FIG. 4. A specific compound is typically added there to bind and detect nickel, such as dimethylglyoxime.

After a sufficient separation has been achieved, the mobile phase may be dried and an eluent may be applied to eluent application regions 124 by placing these regions as well into the same or a different mobile phase in a thin layer chromatography tank. A suitable eluent may flow from eluent application region 124 shown at bottom in FIGS. 3 and 4 via the interconnection regions 125 and may elute the analytes 142 to 145, as indicated by dash-dotted arrows, into the sample interaction regions 126 where an indicator substance for each of the analytes 142 to 145 may be provided. A colorimetric detection therefore indicates whether the analytes 142 to 145, such as cadmium, copper, and lead cations, are present in the sample. The rightmost analyte interaction region 126, or any other analyte interaction region, may also be provided as a negative control channel. This channel may particularly be arranged at a position where it is known that no metals may be detected.

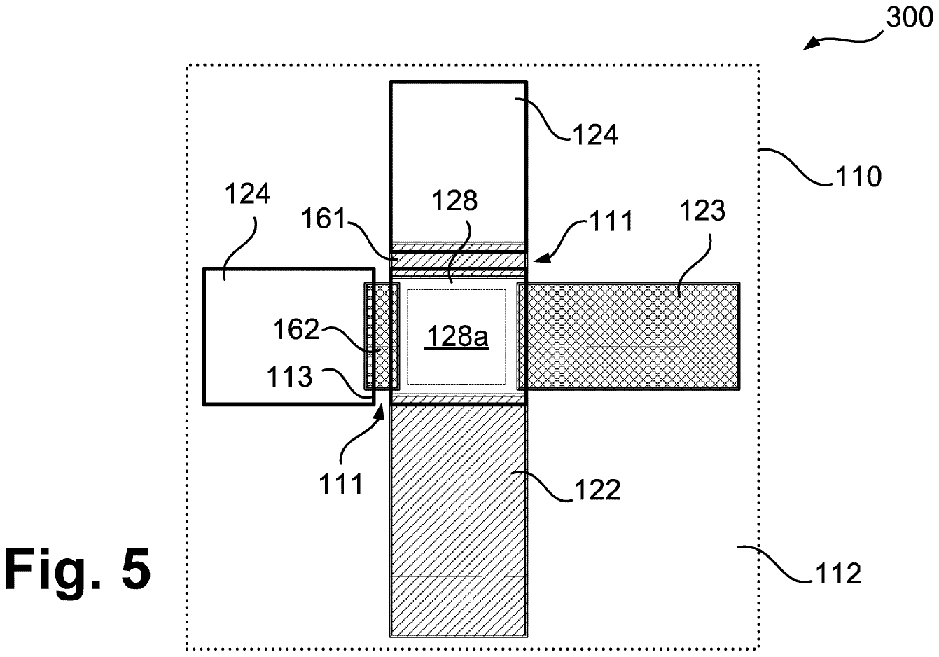

In FIG. 5, an embodiment of device 300 is illustrated which allows for solid phase extraction in a solid phase extraction region 128, i.e., an analytic region. Device 300 includes regions in different planes, wherein, only for reasons of explanation, regions in a common first plane are shown with a solid single-line border and a white filling, regions in a common second plane are shown with a double-line border and a diagonal hatch and regions in a common third plane are shown with a double-line border and a cross-hatch. For clarity, the hatched regions are illustrated as being transparent, such that the borders of the underlying regions shine through, and substrates 110 on which the regions are placed are indicated with a common dotted line. The substrates 110 may have an identical, or different, shapes and are only illustrated for reasons of completeness, without any intended limitation.

In solid phase extraction region 128 in the first plane, a solid phase extraction material is printed or otherwise placed in a region 128a within a frame of a base silica material in a sufficient thickness and with only a slight or no overlap with the frame of the base silica material, and this solid phase extraction material may, for example, include hydrophobically functionalized silica particles or particles of a strong anion exchange resin.

Auxiliary regions in the form of two eluent application regions 124 and an eluent sink region 122 are also provided, as well as a sample or analyte separation region 123 as a further analytic region, and bridging regions 161 and 162 in the second and third plane. As illustrated in FIG. 5, at least the solid phase extraction region 128 or, more precisely, the base material frame, and the two mobile phase application regions 124 are provided as disjoint thin layer regions separated by, e.g., unprinted spaces of the substrate 110 indicated with 111, i.e., strips void of porous material forming diffusion barriers which are, in one instance, indicated with 113. A uncovered space of the substrates 110 forms non-analytic region(s) 112.

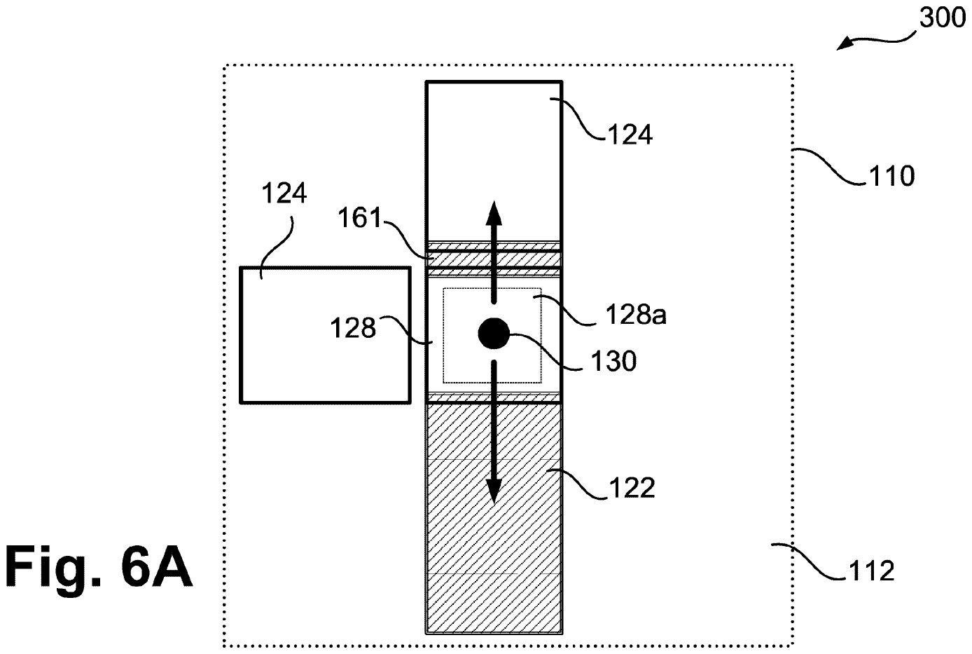

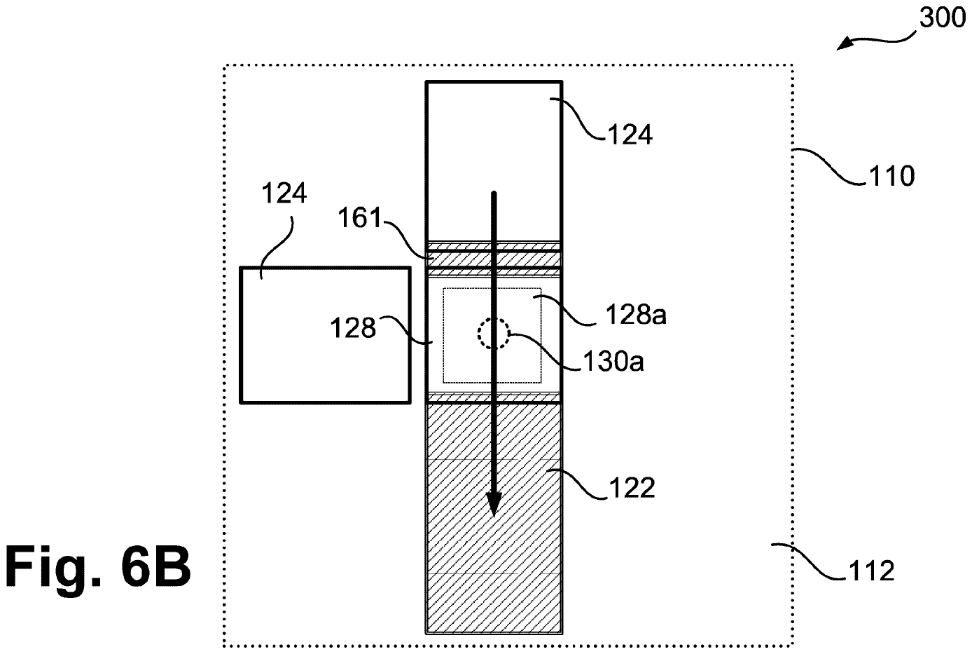

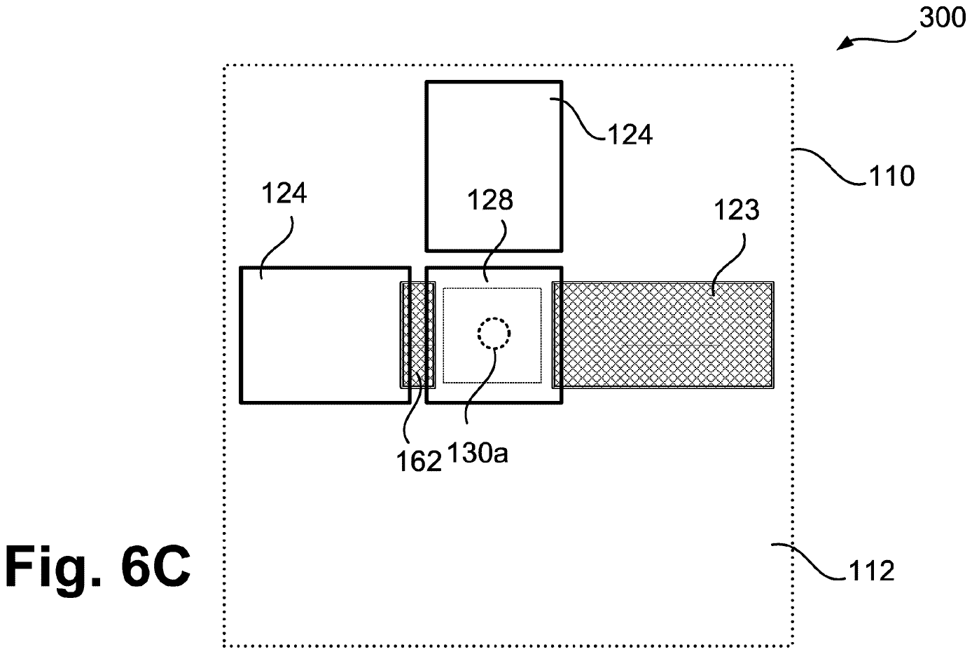

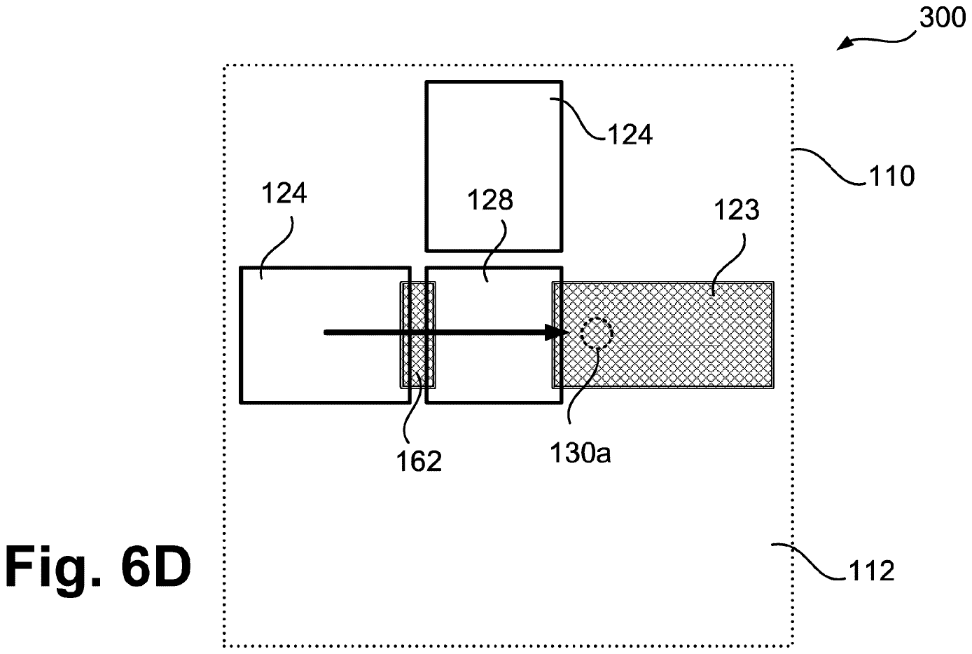

As will be further explained in connection with FIGS. 6A to 6E, the arrangement of the regions shown in FIG. 5 in different layers allows for performing different steps of a solid phase extraction and subsequent chromatographic separation, and each of FIGS. 6A to 6E illustrates a corresponding step. In each of FIGS. 6A to 6E, only those planes of which certain regions are brought into contact with each other in the steps respectively illustrated are shown.

According to a first step shown in FIG. 6A, the solid phase extraction region 128, or more precisely the base material frame thereof, is brought into contact with the eluent application region 124 shown in the upper part of FIGS. 6A to 6E (and FIG. 5) by bridging region 161. Furthermore, eluent sink region 122 is directly brought into contact with an opposite edge of mobile phase application region 128. As illustrated with a solid dot 130 in the centre of solid phase extraction region 128, or its subregion 128a, a sample in a comparatively large volume may be applied, and one or more analytes may bind to, or otherwise interact with, the material in solid phase extraction region 128. Superfluous sample liquid may flow to both the eluent application region 124 shown in the upper part of FIGS. 6A to 6E and the eluent sink region 122, as illustrated with solid arrows pointing upwards and downwards.

As shown in FIG. 6B, in a next step, where the connection configuration is still like that shown in FIG. 6A, an eluent may be added to eluent application region 124 shown in the upper part of FIGS. 6A to 6E to wash interfering compounds from the solid phase extraction region 128 while the analyte(s) are retained by the material in subregion 128a, as generally known in the field of solid phase extraction and as indicated with a dot 130a. The flow of eluent is indicated with an arrow pointing downwards in FIG. 6B.

A next step shown in FIG. 6C results from bringing the eluent application region 124 shown in the upper part of FIGS. 6A to 6E and the solid phase extraction region 128 on the one hand, and the solid phase extraction region 128 and the solid phase sink region 122 on the other hand out of contact by lifting the bridging region 131 and the solid phase sink region 122. Instead, the bridging region 162 is brought into contact with the eluent application region 124 shown in the left part of FIGS. 6A to 6E and the solid phase extraction region 128 and the sample or analyte separation region 123 is also brought into contact with the solid phase extraction region 128.

Using a different (stronger) eluent which is applied to the eluent application region 124 shown in the left part of FIGS. 6A to 6E, as illustrated in FIG. 6D, the analyte(s) 130a retained in solid phase extraction region 128 may now be eluted towards a start of the sample or analyte separation region 123 as illustrated with an arrow pointing right in FIG. 6D. The eluent and its components may be evaporated and/or, if necessary, a neutralization step may be performed.

Using, in a configuration unchanged from that shown in FIG. 6D, the same or a further eluent is applied to the eluent application region 124 shown in the left part of FIGS. 6A to 6E, the analytes may be separated in sample or analyte separation region 123, as illustrated in FIG. 6E with an elongated arrow and with spots 130b and 130c separated accordingly.

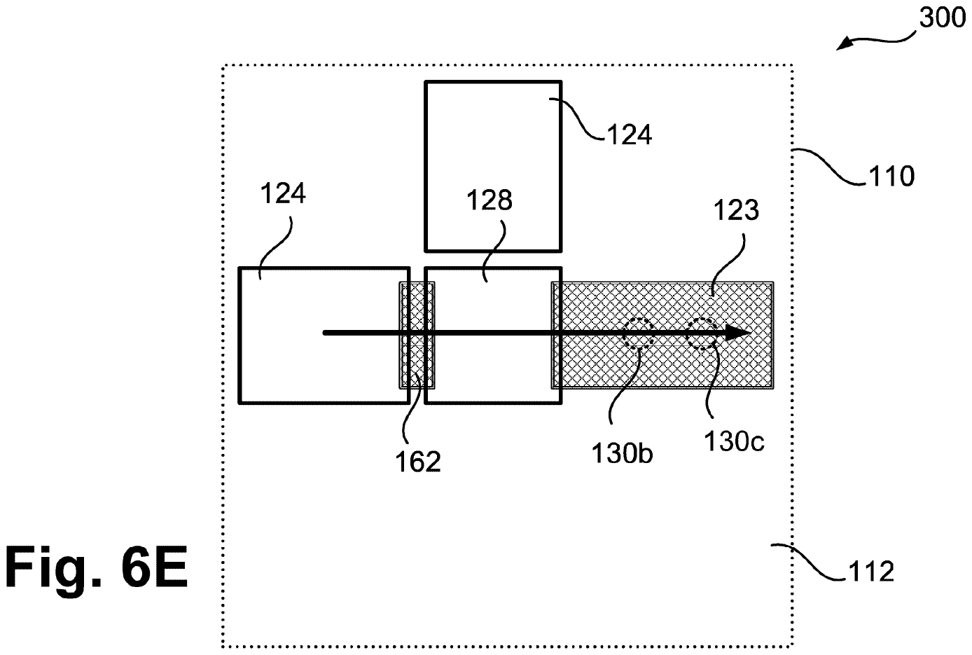

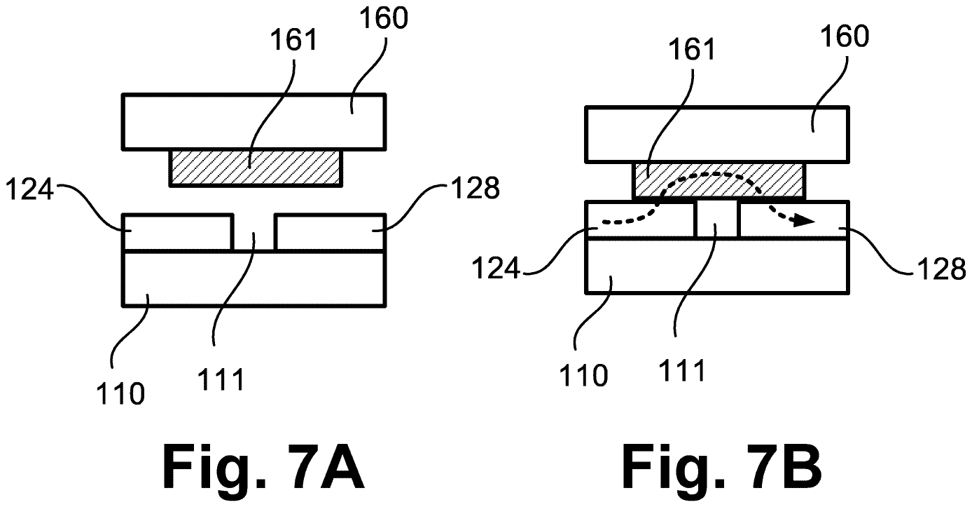

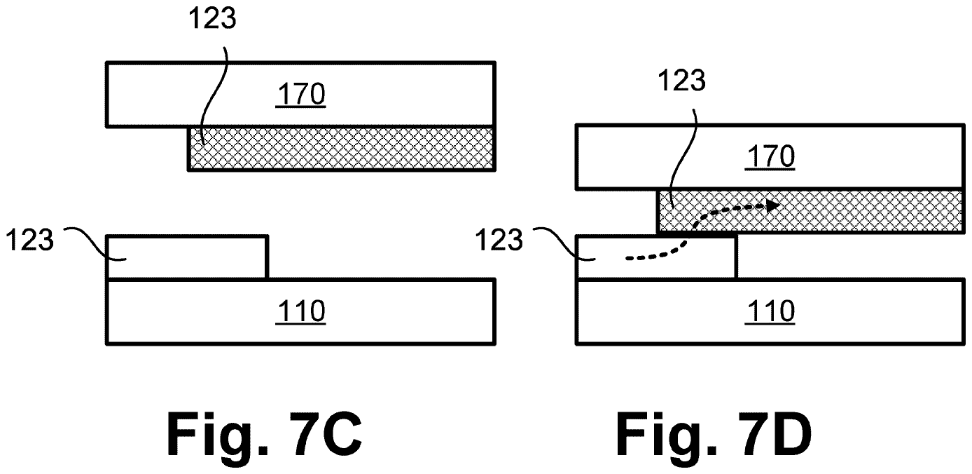

FIGS. 7A and 7B serve to illustrate how, such as in the device 300 shown in FIGS. 5 and 6A to 6E, unprinted spaces 111 may be selectively bridged by using a bridging structure or region, such as the bridging regions 161 and 162 illustrated before, which is brought into contact with exactly the two regions adjacent to the unprinted space 111. In FIGS. 7A and 7B, the bridging region 161 is illustrated but bridging in case of bridging region 162 is performed essentially the same. In the example, regions 124 and 128 are bridged accordingly, as already illustrated in FIGS. 5 and 6A to 6E. A view in FIGS. 7A and 7B corresponds to a vertical section through the plane of the paper in FIGS. 5, 6A and 6B, perpendicular to a longitudinal extension of the bridging region 161 illustrated therein. A substrate is indicated with 110 for the regions 124 and 128 and 160 for region 161, respectively. As can be seen from FIGS. 7A and 7B, the unprinted space 111 is bridged by lowering the bridging structure or region 161, and the connection may be interrupted by raising this region 161. That is, using the bridging structures 161, 162, which may be silica gel layers attached to a substrate 160 as well, which may selectively be lowered in the device 300 and lifted in the same device, as illustrated in FIGS. 7A and 7B, “valves” between the thin layer regions provided as disjoint thin layer regions are provided which may be opened and closed as desired in certain analysis steps. A liquid flow enabled by the closing of the bridge 111 is indicated with a dotted arrow in FIG. 7B.

FIGS. 7C and 7D illustrate how, such as in the device 300 shown in FIGS. 5 and 6A to 6E, solid phase extraction region 128 may be brought into contact with region 122 or region 123. The specific illustration in FIGS. 7C and 7D shows solid phase extraction region 128 together with its substrate 110 and sample or analyte separation region 123, but the connection with mobile phase sink region 122 may be performed analogously. A view in FIGS. 7C and 7D corresponds to a vertical section through the plane of the paper in FIGS. 5 and 6C to 6E, parallel to a longitudinal extension of the 123 illustrated before. As can be seen from FIGS. 7C and 7D, a connection may be simply made by lowering a substrate carrying region 123, and the connection may be interrupted by raising the substrate again.

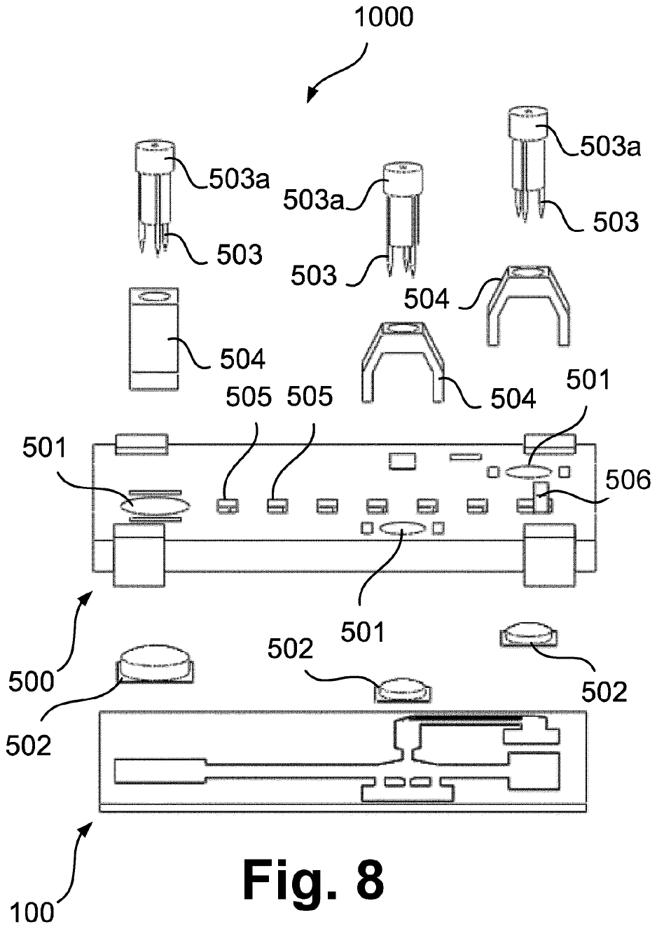

FIG. 8 illustrates an analysis arrangement 1000 according to an embodiment of the present invention including a device 100 as explained before, and a covering structure 500 holding in place, and partly covering, the device 100. It will be noted that device 100 shown in FIG. 8 has a different configuration than that shown in FIG. 1, but arrangement 1000 can be provided for the device shown in FIG. 1. Any other device according to an embodiment of the invention may also be used with a correspondingly adapted covering structure 500. Both the covering structure 500 and the device 100 are shown in a view distorted in perspective, and are illustrated in an exploded view for sake of clarity.

The covering structure 500 comprises receptacles 501 configured to receive liquid reservoirs 502 containing liquids. Liquid reservoirs 502 may be held in place in the receptacles 501. Piercing means 503 configured to pierce the fluid reservoirs 502 upon operating an operating means 503a are associated with the receptacles 501. Guiding means 504 may be provided to guide the piercing means 503 when operated. As the receptacles 501 are placed, in a top view, above certain thin layer regions such as a mobile phase application region 121, and eluent application regions 124, their piercing serves the purpose of applying the corresponding liquids to these regions. That is, the liquid which is contained in the liquid reservoirs 502 is applied to defined regions of the analysis device 100.

The covering structure 500 also may comprise a plurality of cutouts 505, of which only two are indicated for reasons of clarity, which may be provided for the purposes explained before and which may be opened and closed e.g. using a slider mechanism of which an actuator or operating element 506 is shown. At least one of the cutouts 505 may be used for sample application and/or, as mentioned before already, for sample detection.

The covering structure 500 serves to protect device 100 from physical damage and to hold the liquid reservoirs 502 in place. It may also be used to hold and position bridging elements 161, 162 as illustrated in FIGS. 6, 7A and 7B. During a thin layer chromatography separation, the covering structure 500, and more precisely the cutouts 505, is/are preferably essentially closed as otherwise mobile phase may evaporate too fast. Thereafter, for drying, it is preferably opened. Heating from the bottom can also be used to accelerate evaporation.

Materials for liquid reservoirs 502 may be particularly provided as blisters made from aluminium and/or plastics, but may also be provided in a different manner, such as known for tablet blisters. The invention is not limited to a specific type of reservoirs 502, and their material may be any solvent or sample compatible material. These reservoirs 502 may be placed on the printed silica gel regions where the aluminium is in contact with the silica gel. The reservoirs 502 are held in place by the covering structure 500 and the recesses 501. The covering structure 500 particularly presses on the edges of the reservoirs where plastic and aluminium are bonded together.

By acting upon the piercing means 503, which may comprise a plurality of needles attached to an operating element 503a such as a button, the plastic and the aluminium of the liquid reservoirs 502 may be pierced, letting the liquid come into contact with silica gel beneath. As mentioned, a thicker subregions, such as explained for subregions 121a and 124a, may be provided beneath for improving liquid application to the respective regions.

Reservoir materials are particularly selected to be able to tolerate organic solvents, acidic/basic solutions, etc. The plastic material must be pierceable by needles. Aluminium at the bottom, instead of plastics, is particularly advantageous because holes created therein have a smaller tendency to reseal after the piercing elements are retracted. In order to avoid a vacuum forming in the liquid reservoirs 502, the piercing elements may particularly be provided as hollow needles.

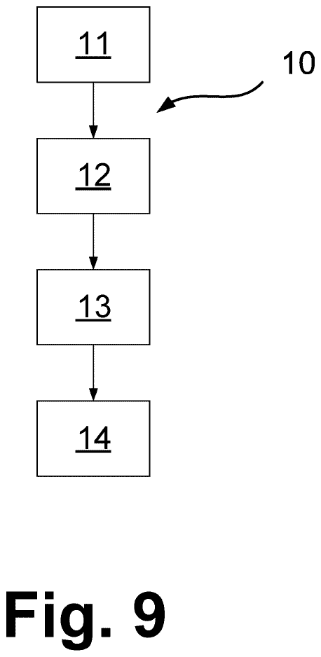

To summarize the above, and as illustrated in FIG. 9 in the form of a method 10, according to an embodiment of the present invention, producing an analysis device for analysing a liquid sample of any of the types mentioned before, comprises the steps of providing 11 a planar substrate and providing 12 thin layer regions on the substrate including screen printing. The method 10 further includes a drying step 13 and a possible washing step 14, or, in other words, a post screen printing treating sequence including drying and washing which was explained before.

1. An analysis device (100, 200, 300) for analysing a liquid sample (130) for one or more analytes (141-145), wherein
the device (100, 200, 300) comprises two or more analytic regions (123, 126, 128a) and one or more non-analytic regions (112);
each of the analytic regions (123, 126, 128a) is provided as a sample interaction region and/or a detection region for the one or more analytes (141-145);
the two or more analytic regions (123, 126, 128a) and the one or more non-analytic regions (112) are provided in one or more substrates in one or more planes;
at least one of the two or more analytic regions (123, 126, 128a) is formed by a porous material made from particles and a binder; and
the two or more analytic regions (123, 126, 128a) being separated from the one or more non-analytic regions (112) by diffusion barriers (113) for fluid in the one or more analytic regions (123, 126, 128a).

2. The device (100, 200, 300) according to claim 1, wherein each of the diffusion barriers (113) is provided as a barrier made from a hydrophobic, non-porous and/or non-polar material or being a boundary between the porous material and a region being void of the porous material.

3. The device (100, 200, 300) according to claim 1, wherein at least one of the two or more analytic regions (123, 126, 128a) is formed by printing the at least one analytic region (123, 126, 128a) onto a substrate (110) or cutting the at least one analytic region (123, 126, 128a) from a sheet of material.

4. The device (100, 200) according to claim 1, wherein any group formed by at least one of the two or more analytic regions (123, 126, 128a) and at least one further region, or a vertical projection thereof, has a concave shape.

5. The device (100, 200) according to claim 4, wherein a longitudinal axis of a first analytic region (123, 126, 128a) is oriented in an acute, right or obtuse angle to a longitudinal axis of a second analytic region (123, 126, 128a).

6. The device (300) according to claim 1, wherein at least one of the two or more analytic regions (123, 126, 128a) is completely enclosed by at least one of the diffusion barriers (113).

7. The device (100, 200, 300) according to claim 1, wherein at least one of the two or more analytic regions (123, 126, 128a) comprises a porous material including one type of particles and at least a different one of the two or more analytic regions (123, 126, 128a) comprises the same or a different porous material including the same or a different type of particles or no particles.

8. The device (100, 200, 300) according to claim 1, wherein at least one of the two or more analytic regions (123, 126, 128a) is provided to interact with at least one component of the liquid sample (130), the at least one component including the analyte (141-145) or at least one of the analytes (141-145).

9. The device (100, 200, 300) according to claim 1, further including one or more supporting regions (121, 122, 124, 125, 127, 161) selected from mobile phase application regions (121), mobile phase sink regions (122), eluent application regions (124), connection regions (125), bridging regions (161), and detection substance application regions (127).

10. The device (100, 200, 300) according to claim 1, wherein
a plurality of further regions (125) is provided to serially or in parallel connect at least two of the analytic regions (123, 126, 128); and/or
at least one of the analytic regions (123, 126, 128a) is provided to connect to at least a further one of the analytic regions (123, 126, 128a) and/or a further region (121,122,124,125,127) via one or more tapered or non-tapered transitions (129) ; and/or
at least one of the analytic regions (123, 126, 128a) is provided in a longitudinal and bent shape to correspond to an area or shape of the substrate (110); and/or
at least one of the analytic regions (123, 126, 128a) is provided to have a greater thickness orthogonal to a surface of a substrate (110) as compared to at least the further one of the analytic regions (123, 126, 128a).

11. The device (100, 200, 300) according to claim 1,
wherein the particles are provided as modified or unmodified silica microcellulose, carbon, and/or polymer based particles; and/or
wherein a binder and/or thickener, particularly xanthan gum, is included in at least one of the analytic regions (123, 126, 128a).

12. An analysis arrangement (1000) comprising the analysis device (100, 200, 300) according to claim 1 and a covering structure (500), the covering structure (500) being configured to position and to at least partly cover the analysis arrangement (100, 200, 300).

13. The analysis arrangement (1000) according to claim 12,
wherein the covering structure (500) comprises at least one of
a receptacle (501) configured to receive a liquid reservoir (502) containing a liquid and piercing means (503) configured to pierce the liquid reservoir (502) upon operating an operating means (503) associated with the receptacle (501); and
at least one cutout (505) for applying the sample (130) and/or a further liquid to the analysis device (100, 200, 300) and/or reading analysis result and/or for providing (2) evaporation control.

14. A method (10) for producing an analysis device (100, 200, 300) for analysing a liquid sample (130) containing one or more analytes (141-145), the method (10) comprising:
providing (11) one or more planar substrates (110) and
providing (12) at least two analytic regions (123, 126, 128a) on the one or more substrates (110);
wherein said providing the two or more analytic regions (123, 126, 128a) includes applying a porous material made from particles and a binder onto the one or more substrates (110).

15. The method (10) according to claim 14, wherein providing the two or more analytic regions (123, 126, 128a) made from particles and a binder comprises screen printing a slurry comprising said particles and said binder onto the substrate (110).

16. A method of analysing a liquid sample (130) containing one or more analytes (141-145), using the analysis device (100, 200, 300) according to claim 1.

17. The method according to claim 16, wherein the analyte or at least one of the analytes (141-145) is at least one of
enriched in at least one of the analytic regions (123, 126, 128a),
chromatographically separated in at least one of the analytic regions (123, 126, 128a),
detected in at least one of the analytic regions (123, 126, 128a), and
transferred between two of the analytic regions (123, 126, 128a).
